(19) United States
(12) Patent Application Publication
(10) Pub. No.: US 2002/0055099 A1
(43) Pub. Date: May 9, 2002

(54) SEQUENTIAL CDNA LIBRARY AND USES THEREOF

(76) Inventor: PAUL B. FISHER, SCARSDALE, NY (US)

Correspondence Address: LISA B. KOLE, BAKER & BOTTS, L.L.P., 30 ROCKEFELLER PLAZA, NEW YORK, NY, 10112, US

(21) Appl. No.: 09/268,302

(22) Filed: Mar. 15, 1999

(51) Int. Cl. C12Q 1/68; C07H 21/02; C07H 21/04

(52) U.S. Cl. 435/6; 536/23.1

(57) ABSTRACT

This invention providing a method of producing a sequential cDNA library array comprising (a) obtaining a subtracted cDNA library having clones containing cDNA inserts and said library also allows propagation of the cDNA inserts (b) arraying the cDNA library into individual clone such that each clone can propagate on its own (c) transferring the arrayed clones onto a solid matrix thereby generating a cDNA library replica (d) extracting the nucleic acid from the clone and (e) generating a subarray of phage clones or DNA inserts. This invention also provides a method of identifying genes comprising hybridizing the cDNA library array with specific probes. The invention also provides the genes identified by the preceeding method. This invention provides a method wherein the sequential cDNA library array is used for diagnostics, genetic screening and prognosis.

Throughout this application, various publications are referenced. The disclosures of these publications in their entireties are hereby incorporated by reference in order to more fully describe the state of the art as known to those skilled therein as of the date of the invention described and claimed herein.

### BACKGROUND OF THE INVENTION

A methodology, Sequential cDNA Library Array (SCLA), is described that permits the identification and cloning of genes displaying differential expression as a consequence of induction of growth suppression, reversible differentiation, terminal differentiation and apoptosis. This scheme has wide applicability for systematically evaluating and identifying cDNAs in a subtracted library displaying differential expression as a function of complex changes in cell physiology. The SCLA approach will be beneficial for studying a large number of intricate biological processes and it offers potential for identifying and cloning genes providing diagnostic, prognostic and therapeutic utility.

Changes in the expression of specific subsets of genes, including induction, suppression and modulation, underlie many important aspects of cellular physiology. In most instances, alterations in the amount of specific mRNA populations profoundly contribute to consequential processes such as growth regulation, terminal differentiation, cellular senescence and development. Moreover, aberrant gene expression is a major contributor to many disease states, including cancer and developmental abnormalities. On the basis of these considerations, defining the temporal spectrum of gene changes associated with and potentially controlling cellular fate is paramount to understanding and potentially intervening in and modifying critical cellular processes such as aging, cancer, development, differentiation and growth.

The ability to identify and clone differentially expressed genes is achievable using several methodologies, including subtractive hybridization (Jiang and Fisher, 1993; Sagerström et al., 1997; Huang et al. 11999), differential RNA display (DDRT-PCR) (Liang and Pardee, 1992), RNA fingerprinting by arbitrarily primed PCR (RAP-PCR) (Ralph et al., 1993; McClelland et al., 1995), suppression subtractive hybridization (Diatchenko et al., 1996), representational difference analysis (RDA) (Hubank and Schatz, 1994), serial analysis of gene expression (SAGE) (Velculescu et al., 1995; Zhang et al., 1997), electronic subtraction (Adams et al., 1993; Wan et al., 1996), combinatorial gene matrix analysis (Lockhart et al., 1996) and reciprocal subtraction differential RNA display (RSDD) (Kang et al., 1998b). Some of these schemes are technically difficult and others suffer from problems including, the cloning of short DNA sequences (representing only minimal gene regions and rarely containing a complete open reading frame) (Liang and Pardee, 1995; McClelland et al., 1995; Lockhart et al., 1996; Hubank and Schatz, 1994; Diatchenko et al., 1996; Velculescu et al., 1995; Kang et al., 1998b), the identification of a significant number of false positive cDNAs (Liang and Pardee, 1995; McClelland et al., 1995), the requirement for sophisticated equipment (not accessible to the majority of researchers) and prohibitive costs for those in academic laboratories (Lockhart et al., 1996; Adams et al., 1993; Velculescu et al., 1995; Schena et al., 1995; Sagerström et al., 1997; Wan et al., 1996) (Table 1). In addition, evidence has been provided that in specific instances, such as RSDD (Kang et al., 1998(b)) which employs reciprocal subtraction of cDNA libraries followed by DDRT-PCR, the differentially expressed genes that are isolated are often different than those obtained using subtraction hybridization or DDRT-PCR independently. A further complexity in isolating differentially expressed genes results because the majority of genes display distinct temporal kinetics of expression. In these contexts, it may be necessary to use a range of approaches that incorporate a step permitting the isolation of temporally expressed cDNAs to identify and clone a complete array of differentially expressed genes (Jiang and Fisher, 1993; Huang et al., 1999).

As emphasized, to discern the molecular basis of complicated processes such as differentiation, senescence and apoptosis it will be important to define the complete repertoire of genes associated with and potentially regulating these phenomena. In HO-1 human melanoma cells, treatment with a combination of recombinant human fibroblast interferon (IFN-β) and the protein kinase C (PKC) activating agent mezerein (MEZ) results in irreversible growth arrest, extinction of oncogenic potential in nude mice and terminal cell differentiation (Fisher et al., 1985; Jiang and Fisher, 1993; Jiang et al. 1994). In contrast, exposure of HO-1 cells to either agent alone results in a reversible inhibition of growth and the reversible induction of specific markers of melanoma differentiation (Fisher et al., 1985; Jiang et al., 1993). Growth of HO-1 cells in medium containing retinoic acid induces melanin synthesis, a marker of melanoma differentiation, without altering cell growth and the combination of IFN-β and recombinant immune interferon (IFN-γ) reversibly inhibits growth without inducing differentiation (Jiang et al., 1993; Graham et al., 1991). Moreover, treatment of HO-1 cells with staurosporine, a cell permeable protein serine/threonine kinase inhibitor (a potent inhibitor of protein kinase C) (Tamaoki et al., 1986), induces apoptosis. For these reasons, the HO-1 human melanoma system represents an ideal model for defining the programs of gene expression changes involved in regulating cellular pathways associated with growth, reversible differentiation, terminal differentiation and apoptosis.

An efficient methodology is presently described, Sequential cDNA Library Array (SCLA), that permits the systematic identification and cloning of a spectra of differentially expressed genes associated with and potentially mediating discrete physiological pathways, such as growth regulation, terminal differentiation and apoptosis, in mammalian cells. SCLA comprises the generation of subtracted cDNA libraries, the isolation and arraying of the entire population of phages potentially containing differentially expressed cDNAs, immobilization of phage on a matrix (such as nylon filters) and hybridizing with pooled mRNAs isolated from target cells (reverse Northern analysis). Positive hybridizing clones can easily be identified, isolated from the primary arrayed phages and characterized, including sequencing, isolation of a complete open reading frame and functional analysis. To enhance and insure the isolation of cDNAs representing genes displaying complex temporal kinetics, pooled mRNAs are isolated from target cells that span the time course experimentally determined to be involved in the phenotypic change under investigation. By probing replicate phage containing filters with defined mRNA populations, SCLA permits the identification of both pathway (agent) unique, overlapping and competitive gene expression changes.

It is hypothesized that genes associated with induction and maintenance of terminal differentiation may also contribute to additional growth related phenomenon, including cell cycle control, senescence and apoptosis. This possibility has now been experimentally assessed using the HO-1 model induced to terminally differentiate by treatment with IFN-β+MEZ. By probing SCLA clones from a temporally spaced IFN-β+MEZ-treated HO-1 subtracted cDNA library with mRNAs from HO-1 cells temporally treated with IFN-β, MEZ, IFN-β+MEZ or staurosporine, we have now identified a subset of cDNAs that are independently and coordinately regulated by the different experimental conditions. These include cDNAs that may contribute to or associate with growth control, reversible differentiation, terminal differentiation and apoptosis.

The SCLA approach will find wide application for the rapid and efficient identification and cloning of differentially expressed cDNAs. These include, genes expressed at higher or lower levels in normal versus cancer cells, genes differentially expressed during many important cellular processes (such as cell growth, differentiation, senescence, development and apoptosis), genes differentially expressed as a function of treatment with chemotherapeutic agents and viral infection, and genes involved in specific clinically relevant states (such as neurodegeneration, cardiac dysfunction, angiogenesis, muscular degeneration and inborn genetic anomalies). In summary, SCLA will be of benefit for scientists concerned with defining complex and important biochemical pathways regulating cellular physiology and homeostasis within the context of the experimental model being manipulated and studied.

### SUMMARY OF THE INVENTION

The present invention provides a method of producing a sequential cDNA library array comprising: (a) obtaining a subtracted cDNA library having clones containing cDNA inserts and said library also allows propagation of the cDNA inserts; (b) arraying the cDNA library into individual clones such that each clone can propagate on its own;(c) transfering the arrayed clones onto a solid matrix thereby generating a cDNA library replica (d) extracting the nucleic acid from the clone and (e) generating a subarray of phage clones or DNA inserts. In accordance with the method of this invention, the solid matrix may comprise a nylon, nitrocellulose, glass, plastic or DEAE-cellulose membrane. In accordance with this invention, more than one array may be produced. In accordance with this invention the cDNA library may be a λZAP subtracted cDNA library or any subtracted cDNA library.

Further, the present invention provides A method of identifying genes comprising hybridizing the cDNA library array with specific probes. In accordance with this invention the specific probes may be derived from a target cells, tissues, organs or an organism and the probes may be RNA or DNA. In accordance with this invention the target cell is cancerous, embryonic cells, adult cells, infected cells or drug treated cells. In accordance with this invention specific probes are derived from a treatment inducing a specific cellular change or pathway and the pathway or cellular change is growth regulation, terminal differentiation, apoptosis, senescence, neurodegeneration, cardiac dysfunction, angiogenesis, muscular degeneration or treatment induced and the treatment is with an infectious agent or a chemotherapeutic agent. In accordance with this invention the treatment with infectious agents is with a virus, bacteria, fungus or parasite. This invention further provides that the pathway or cellular change is a biochemical pathway and is induced by environmental changes or therapeutic changes.

This invention further provides that the subtracted cDNA library is derived from the HO-1 melanoma cells. In accordance with this invention the melanoma cells are HO-1 melanoma cells treated with IFN-β plus MEZ or untreated HO-1 melanoma cells.

In one embodiment of the invention the invention identifies a gene. In accordance with this invention the gene encodes a protein. Further still, the present invention provides an antibody capable of specifically recognizing the protein.

In the practice of this invention the isolated nucleic acid molecule may be designated Differentiating Melanoma Arrayed Clone (DMAC)-1 thru DMAC-427.

In a further embodiment of the invention the sequential cDNA library array is used for diagnosis, genetic screening or prognosis. In accordance with this invention the diagnosis, genetic screening or prognosis is between cell or tissue types or between species.

### BRIEF DESCRIPTION OF THE FIGURE

FIG. 1

Schematic of the Sequential cDNA Library Array technology. In this outline, SCLA is used to identify genes differentially expressed in a temporally spaced subtracted IFN-β+MEZ-treated HO-1 cDNA library after treatment with IFN-β+MEZ, MEZ, IFN-β or Staurosporine. For this application of the SCLA approach two temporally spaced cDNA libraries are constructed. Subtraction hybridization is then performed, phages are plated on bacterial lawns and individual phage populations potentially containing cDNA inserts are isolated and arrayed (for present and future applications). Replicate arrays are immobilized on nylon filters and hybridized (reverse Northern blotting) with mRNAs from HO-1 cells exposed to the various treatment protocols. A black circle indicates positive hybridization and an open circle indicates negative hybridization.

FIG. 2

A representative application of the SCLA approach to define genes specifically up-regulated in arrayed phage clones isolated from a subtracted cDNA library. The data presented is an autoradiogram of reverse Northern hybridization of a phage replica membranes (representing one array plate containing 384 phage isolates) probed with mRNAs from IFN-β+MEZ, MEZ, IFN-β or Staurosporine treated HO-1 cells. Nylon membranes of phage replicas (derived from the arrayed phage population isolated from a temporally spaced subtracted IFN-β+MEZ-treated HO-1 cDNA library) were probed with ³²P-labeled cDNA reverse transcribed from mRNAs isolated from HO-1 cells treated for 2, 4, 6, 8, 12, 24 and 48 hr with IFN-β+MEZ (2000 units/ml+10 ng/ml), MEZ (10 ng/ml) or IFN-β (2000 units/ml) or treated for 4, 8 and 24 hr with Staurosporine (500 ng/ml). The replica membranes were hybridized with the probes indicated. The hybridized membranes were washed and exposed to Kodak X-OMAT film.

### DETAILED DESCRIPTION OF THE INVENTION

The present invention provides a method of producing a sequential cDNA library array comprising: (a) obtaining a subtracted cDNA library having clones containing cDNA inserts and said library also allows propagation of the cDNA inserts; (b) arraying the cDNA library into individual clones such that each clone can propagate on its own; (c) transfering the arrayed clones onto a solid matrix thereby generating a cDNA library replica (d) extracting the nucleic acid from the clone and (e) generating a subarray of phage clones or DNA inserts.

In an embodiment the solid matrix is a membrane. In another embodiment the membrane includes but is not limited to nylon, nitrocellulose, glass, plastic or DEAE-cellulose.

In a further embodiment at least more than one array is produced.

In an embodiment, the cDNA library may be a λZAP subtracted cDNA library or any subtracted cDNA library.

An embodiment of this invention provides a method of identifying genes comprising hybridizing the cDNA library array with specific probes. In an embodiment of this invention the probes are RNA or DNA. In a further embodiment the specific probes are derived from target cells, tissues, organs, or an organism. In a further embodiment the specific probes are derived from a pathway or cellular change. In an embodiment the cellular change or pathway is growth regulation, terminal differentiation, apoptosis, senescence, neurodegeneration, cardiac dysfunction, angiogenesis or muscular degeneration pathway or treatment induced and the treatment is with an infectious agent or a chemotherapeutic agent. In an embodiment the infectious agent is a virus, bacteria, fungus or a parasite. In a further embodiment the cellular change or pathway is a biochemical pathway.

In an embodiment the subtracted cDNA library is derived from the HO-1 melanoma cells. In a further embodiment the melanoma cells are HO-1 melanoma cells treated with IFN-β plus MEZ. In another embodiment the melanoma cells are untreated.

In one embodiment of the invention the invention identifies a gene. In an embodiment the gene encodes a protein. In a further embodiment this invention provides an antibody capable of specifically recognizing the protein.

In an embodiment of this invention the isolated nucleic acid molecule may be designated Differentiating Melanoma Arrayed Clone (DMAC)-1 thru DMAC-427.

The present invention relates to the production of a sequential cDNA library array which is directed to a methods of comparing one RNA source with another. Such methods may be useful for screening in disease states. In one embodiment of the invention, cancerous cells are monitored to determine stages of development. In the preferred embodiment monitoring is of the gene changes associated with cellular process such as aging, cancer, development, differentiation and growth.

The practice of the present invention may provide systematic identification and cloning of a spectra of differentially expressed genes associated with growth regulation, terminal differentiation and apoptosis.

More specifically, methods which are well known to those skilled in the art can be used to construct a sequential cDNA library array. These methods include in cell culture techniques, RNA extraction, cDNA subtraction and hybridization, amplification and sequencing. See e.g., the techniques described in Maniatis et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y.

Experimental Details

Materials and Methods

Cell Culture and Treatment Protocols

Human HO-1 melanoma cells were grown in Dulbecco's modified Eagle' medium supplemented with 10% fetal calf serum, penicillin/streptomycin (100 U/100 μg/ml) at 37° C. in a 5% CO₂ air humidified incubator (Fisher et al., 1985). Cells were refed with fresh growth medium and treated with 2,000 U/ml IFN-β, 10 ng/ml of MEZ in DMSO (final 0.1% DMSO), 2,000 U/ml IFN-β+10 ng/ml MEZ in DMSO (final 0.1% DMSO) or 500 ng/ml staurosporine.

RNA Extraction, cDNA Library Construction and Subtraction Hybridization

Control and HO-1 cells treated with IFN-β, MEZ or IFN-β+MEZ were harvested at 2, 4, 8, 12, 24 and 48 hrs for RNA extraction. Staurosporine-treated HO-1 cells were harvested at 4, 8 and 24 hrs after treatment. Total RNA was prepared by lysis in 4 M guanidine isothiocyanate followed by ultracentrifugation through CsCl gradients (Sambrook et al., 1989). For temporally spaced cDNA libraries, control and IFN-β+MEZ-treated RNA samples from each time point were pooled and mRNA was purified by oligo (dT)-cellulose column chromatography (Sambrook et al., 1989). Control (DRIVER) and IFN-β+MEZ-treated (TESTER) temporally spaced cDNA libraries were constructed with a λZAP cDNA library kit as described by the manufacturer (Stratagene). A subtracted λZAP cDNA library (IFN-β+MEZ-treated minus control) was constructed as described previously (Jiang and Fisher, 1993).

Array of the Subtracted cDNA Library in 384-well Microtiter Plates and Replicate Phage Plating

Both prior to and after amplification, the subtracted cDNA library was plated at ˜500 plaques/15-cm bacterial culture plate to ensure good separation. Each plaque was manually isolated (by toothpick) and transferred to an individual well in a 384-well microtiter plate containing 50 μl SM-1 μl chloroform/well (primary working array) and stored at 4° C. This approach produced a primary un-amplified and a primary amplified working array. Additional secondary and tertiary arrays of the un-amplified and amplified subtracted cDNA library in 384-well plate containing 50 μl SM with 7% DMSO/well was prepared and stored at minus 80° C. for long term storage. After isolation, the remaining phage plaques were harvested from the plate for further applications including amplification. Arrayed un-amplified and amplified phages were transferred onto nylon membranes with a 384-pin replicator and grown on the bacterial lawn. Phage replicas on nylon membranes were lyzed, denatured, neutralized, washed and UV-crosslinked using standard protocols for further analysis (Sambrook et al., 1989).

Reverse Northern Analysis

For reverse Northern analysis, RNA samples from each time point were pooled and 20 μg of pooled total RNA was reverse transcribed in two reactions into a ³²P-labeled first strand cDNA probe. After heating at 70° C. for 10 min and quenching on ice for two min, a mixture consisting of 10 pmole of T₁₃V and 10 μg of the total RNA mixture in 50 mM Tris-HCl (pH 8.3), 75 mM KCl, 3 mM MgCl₂, 10 mM DTT, 0.5 mM DATP, 0.5 mM dGTP, 0.5 mM dTTP, 0.02 mM dCTP, 5 U RNase inhibitor (Gibco BRL), 100 μCi dCTP (3000 Ci/mmole from Amersham) and 200 U Superscript RT II (Gibco BRL) was added (final 25 μl). The reaction mixture was incubated at 42° C. for one hr and at 37° C. for 30 min after addition of 2 μl of RNase H (10U, Gibco BRL). The membrane was hybridized at 42° C. overnight in a 50% formamide hybridization solution. The hybridized membrane was washed 2× at room temperature for 15 min with 2× SSC containing 0.1% SDS and then washed for at least one hr at 55° C. with 0.1× SSC containing 0.1% SDS. The membranes were exposed for different times to autoradiography.

Results and Discussion

HO-1 Human Melanoma Cells as a Model System to Study Growth, Differentiation and Apoptosis.

Induction of terminal differentiation in HO-1 cells is coupled with a number of distinct alterations in cellular phenotype and gene expression (Fisher et al., 1985; Jiang et al., 1993; Jiang and Fisher, 1993; Jiang et al., 1994; Huang et al., 1999). The biochemical and cellular changes induced in this model cell culture system during terminal differentiation include irreversible growth suppression, an elevation in melanin synthesis (biochemical differentiation), discrete changes in cellular morphology (morphologic differentiation), and modifications in antigenic properties (immunological differentiation) (Fisher et al., 1985; Graham et al., 1991; Jiang et al., 1993, 1994). The identification of specific agents that induce growth inhibition and discrete differentiation changes, in a reversible or irreversible manner, in human melanoma cells documents that this model will have utility for identifying genes associated with and potentially mediating these complex changes in cellular properties induced by the different treatment protocols (Jiang et al., 1993, 1994).

A random sampling of cDNA clones isolated from a temporally spaced IFN-β+MEZ-treated HO-1 subtracted library identified both known and novel genes of physiological relevance to cell growth, differentiation and apoptosis (Jiang and Fisher, 1993; Jiang, et al., 1994). In a more systematic approach for identifying genes of potential importance to terminal differentiation we have used a high throughput microchip cDNA (Synteni; Schena et al., 1995) array screening approach (Huang et al., 1999). An analysis of 1000 cDNA clones from a temporally exposed differentiation inducer treated subtracted (DISH) library resulted in the identification and cloning of 26 known and 11 novel cDNAs implicated in the processes of growth regulation and differentiation (Huang et al., 1999). This approach could only be used to evaluate cDNAs of >500 bp and provided an analysis of 1,000 cDNA clones representing ˜10% of the DISH cDNA library. However, based on the diversity of relevant genes that have already been identified using this scheme, further studies using this type of approach are warranted. Moreover, these results support the value of DISH cDNA libraries for defining the genes associated with and potentially causally related to the phenotypic changes induced in human melanoma cells when they irreversibly growth arrest, terminally differentiate and exhibit a suppression in oncogenic potential in nude mice.

Treatment of HO-1 cells with IFN-β (2000 units/ml) or MEZ (10 ng/ml) results in growth suppression and the induction of specific markers of differentiation (Fisher et al., 1985; Jiang et al., 1994). In the case of IFN-β treatment, HO-1 cells display an elevation in melanin synthesis. With MEZ treatment, HO-1 cells produce elevated melanin levels and exhibit morphological differentiation (characterized by the presence of dendrite-like structures). Upon removal of these agents, growth returns to normal levels and markers of differentiation are extinguished. When treated with IFN-β+MEZ, HO-1 cell growth is inhibited irreversibly, melanin levels are elevated and cellular morphology is altered. In addition, profound changes in gene expression occur in HO-1 cells during the initiation and maintenance phases of terminal differentiation (Jiang et al., 1993, 1994; Kang et al., 1998a). These include, an elevation in expression of c-jun, jun-B, HLA Class I, p21 (mda-6), mda-7, interferon inducible (ISGF-3, ISG-15, ISG-54, 6-16, 71 kD 2′5′A synthetase, 56 K protein, p78 and mda-9), heat shock protein 70, clk-2 kinase, gro/MGSA, α₅ integrin, β₁ integrin, fibronectin and 13 novel cDNAs (Jiang et al., 1993, 1994, 1995a, 1995b; Lin et al., 1998). A decrease in expression of specific genes also occurs during the differentiation process in HO-1 cells, including c-myc, cyclin A, cyclin B, tenascin, α-actin, β-actin, cdc 2, histone H1 and histone H4 (Jiang et al., 1993, 1994, 1995c). As discussed briefly above, since all of the cDNA clones in the subtracted cDNA library have not been isolated or rigorously analyzed, it is highly probable that a significant number of genes involved in growth, differentiation and cancer reversion remain to be identified and cloned.

Identification by Means of the SCLA Technology of Agent (and Pathway) Specific Gene Expression Changes Corresponding with Growth Inhibition, Reversible Differentiation, Terminal Differentiation and Apoptosis.

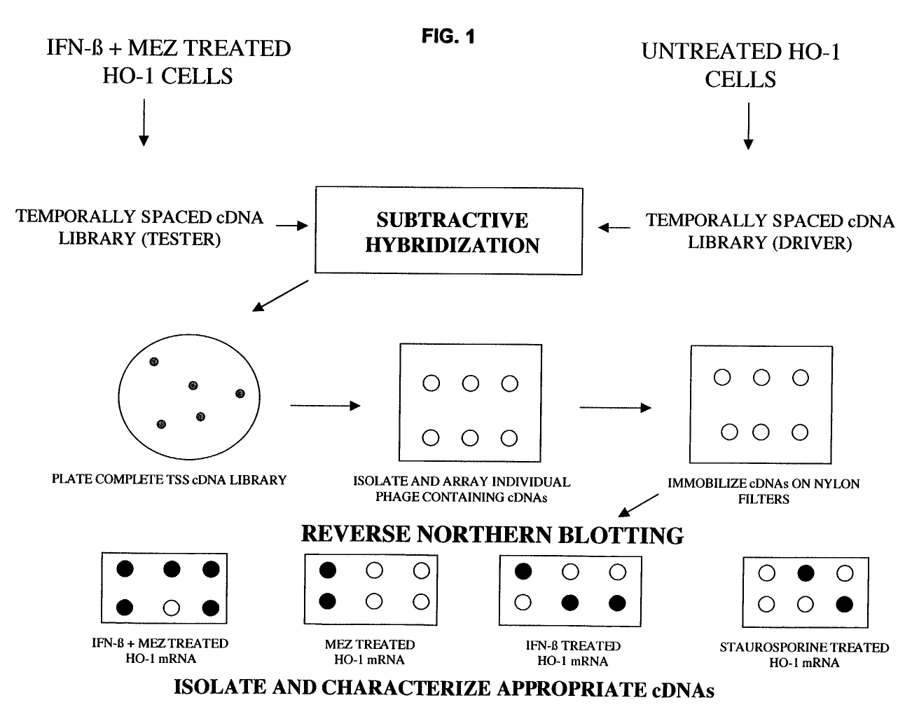

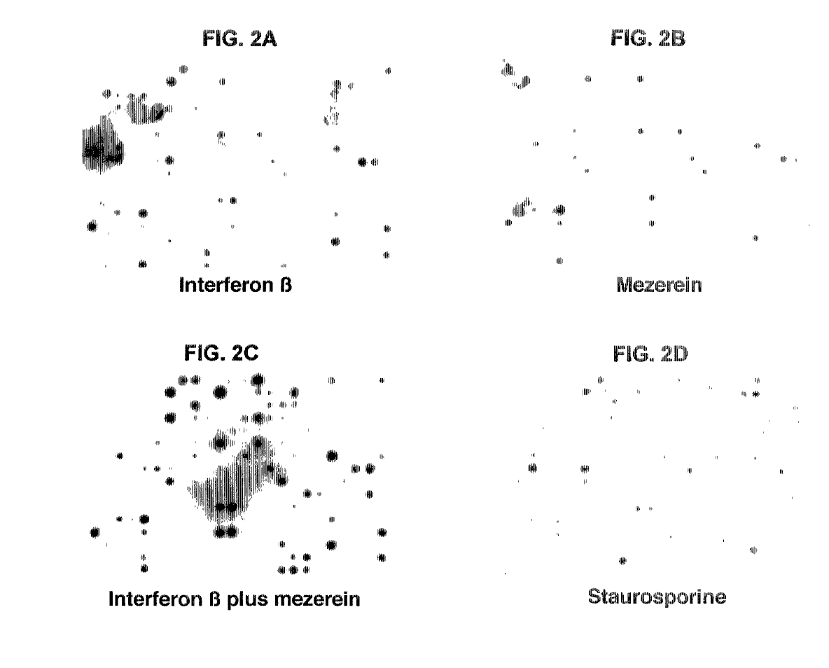

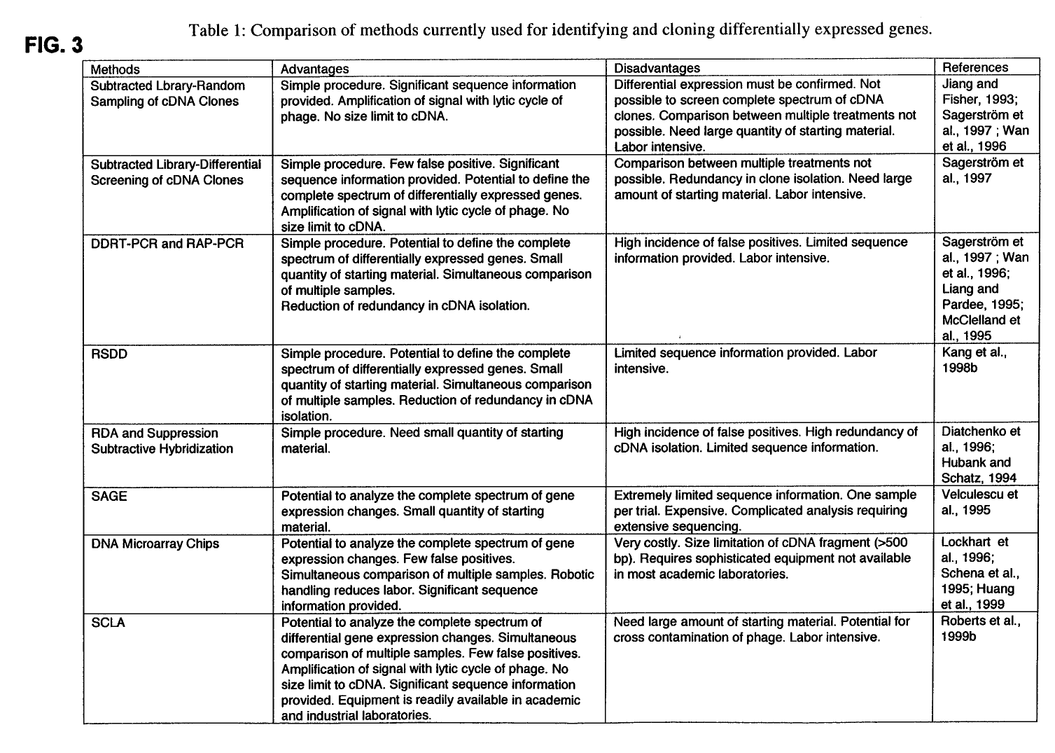

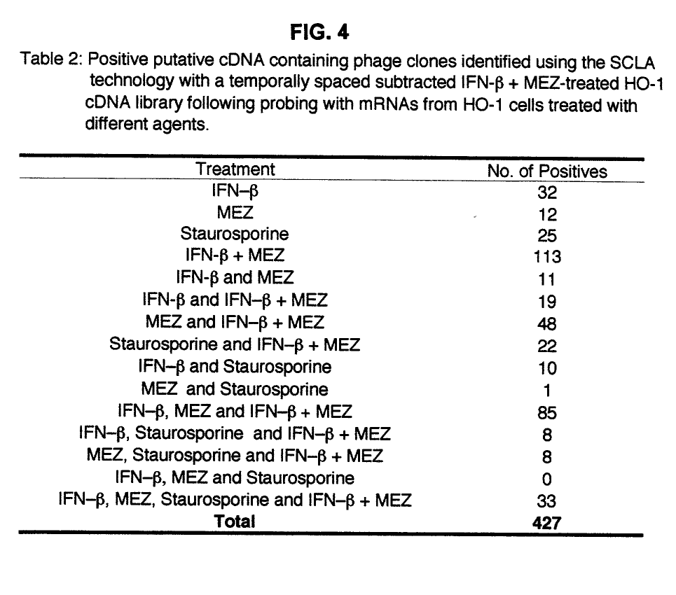

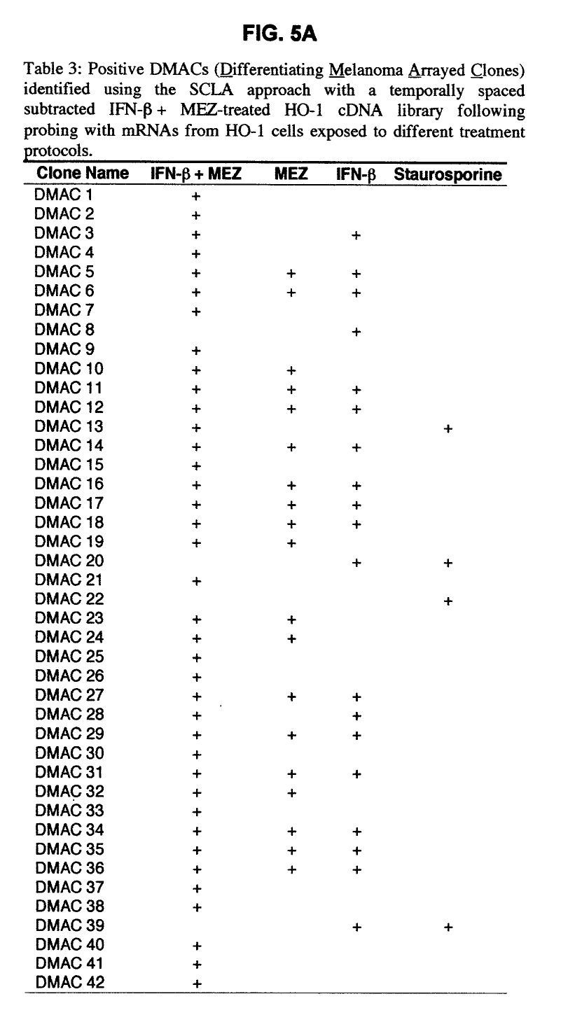

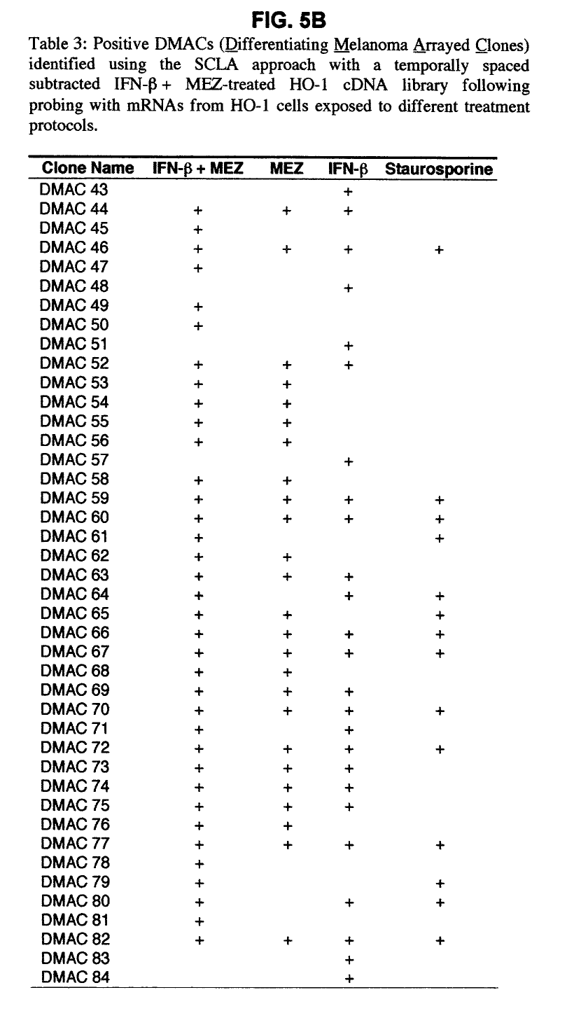

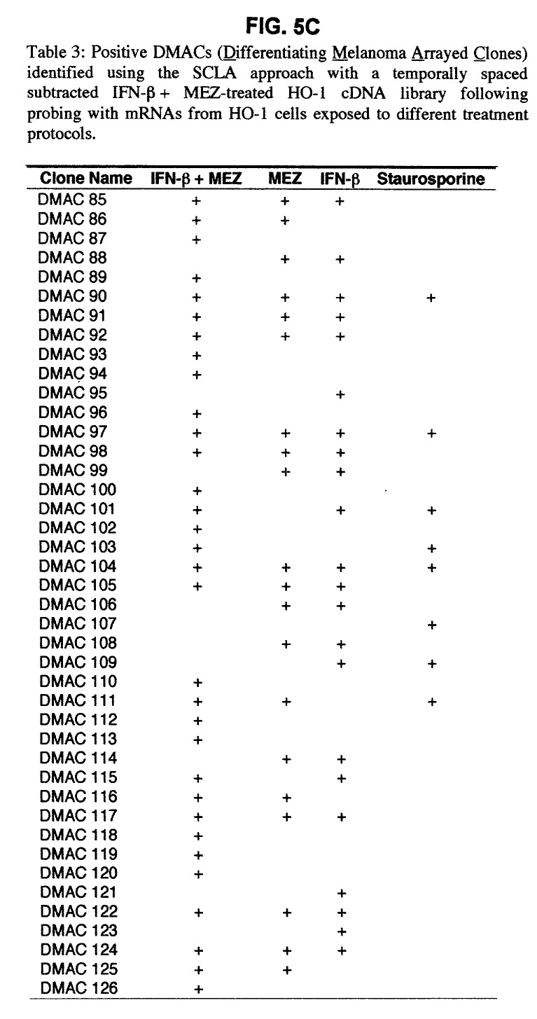

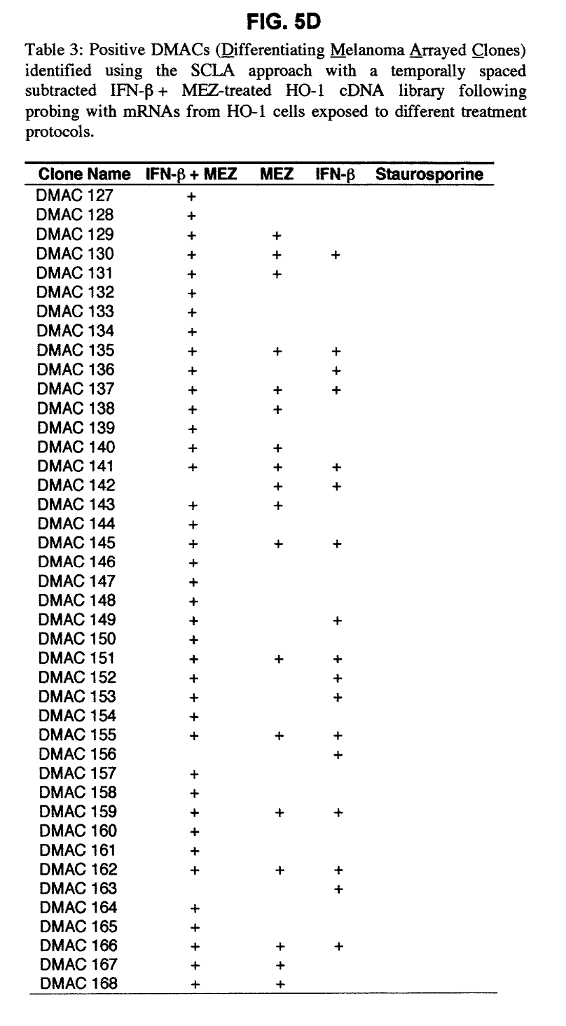

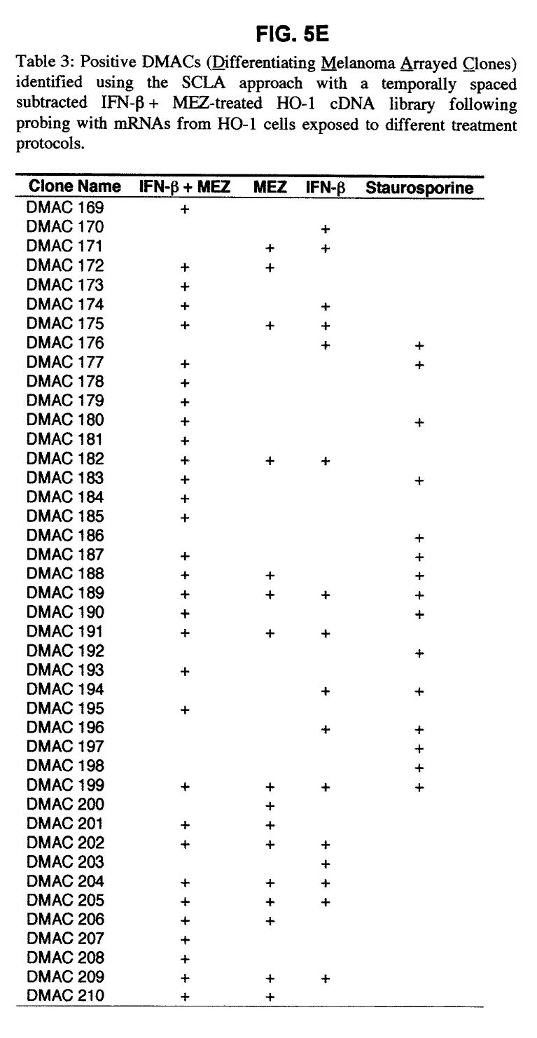

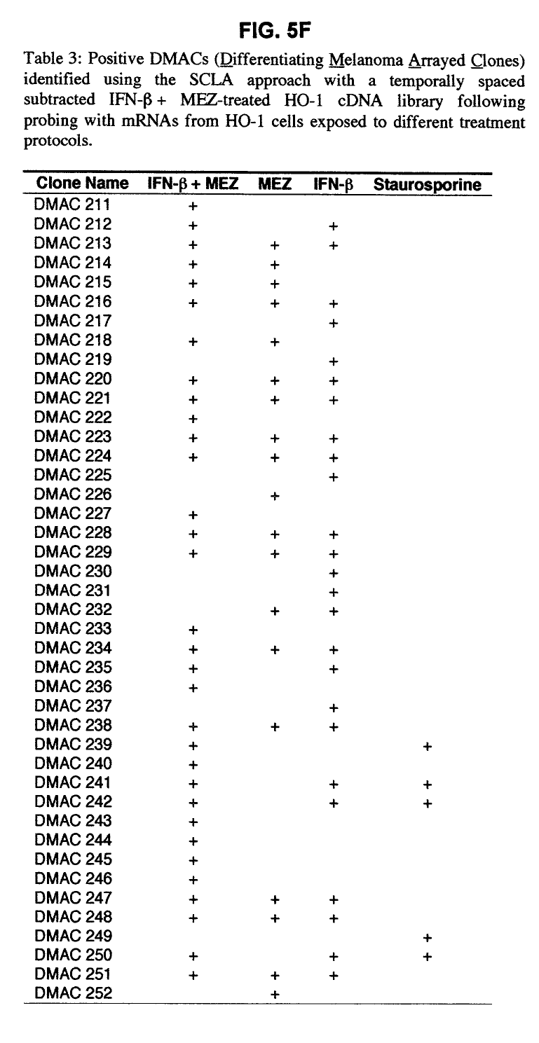

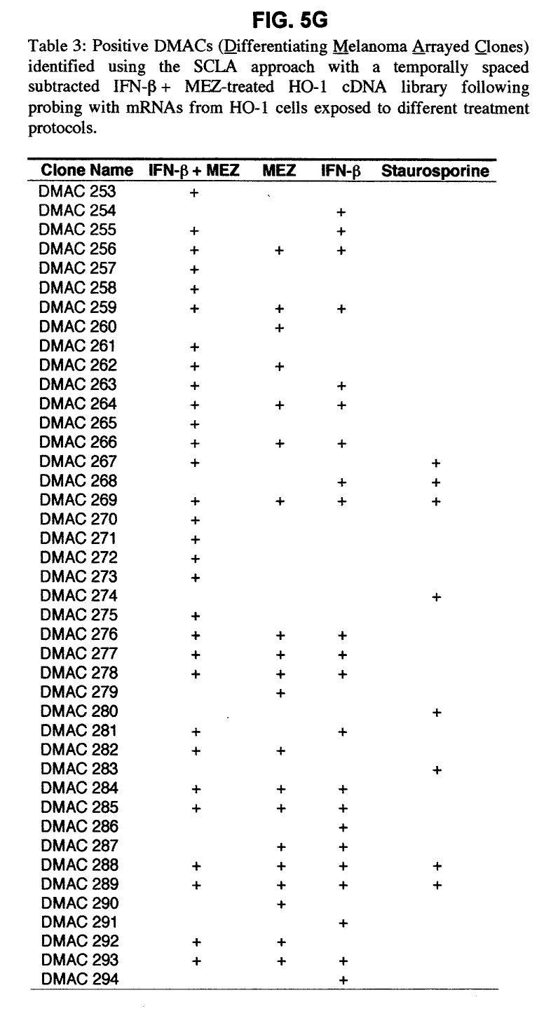

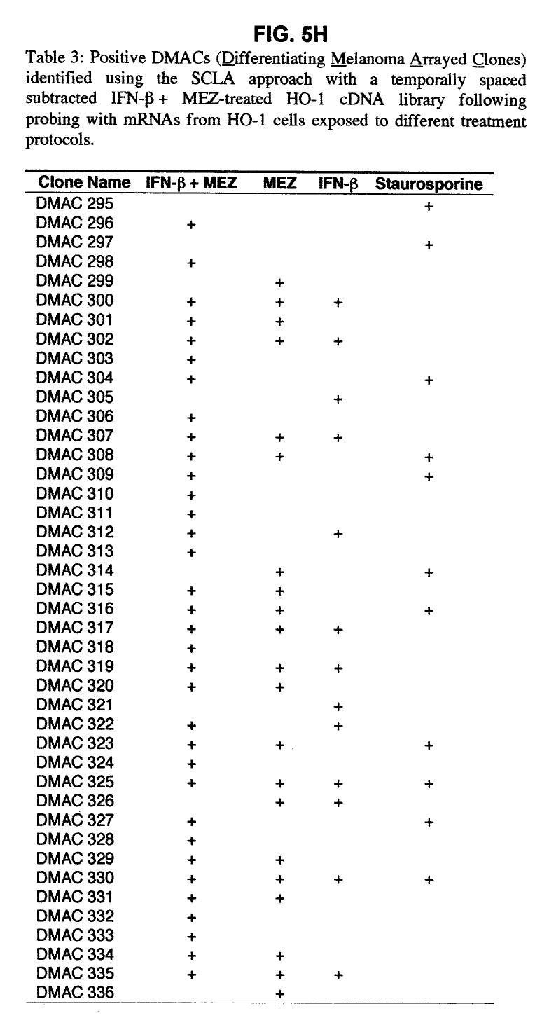

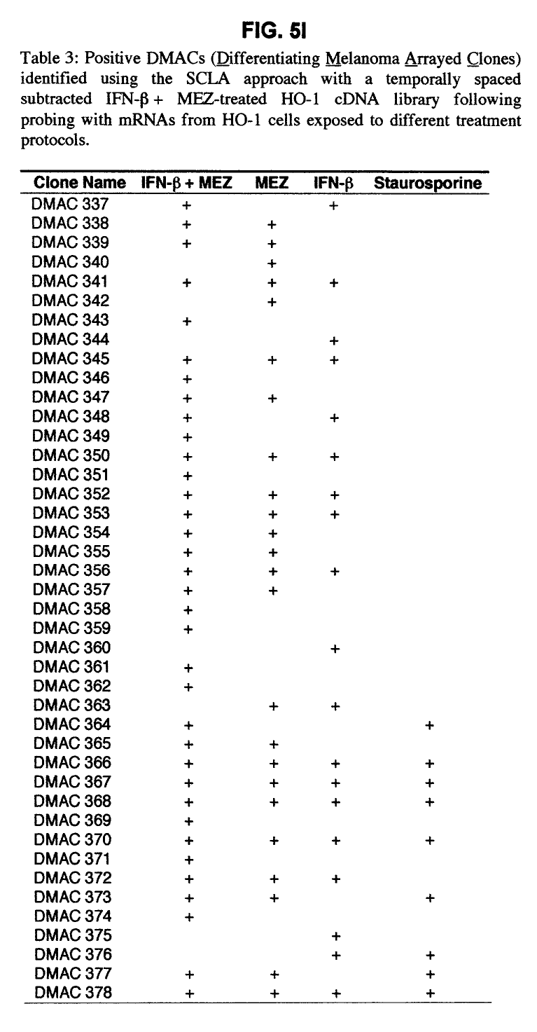

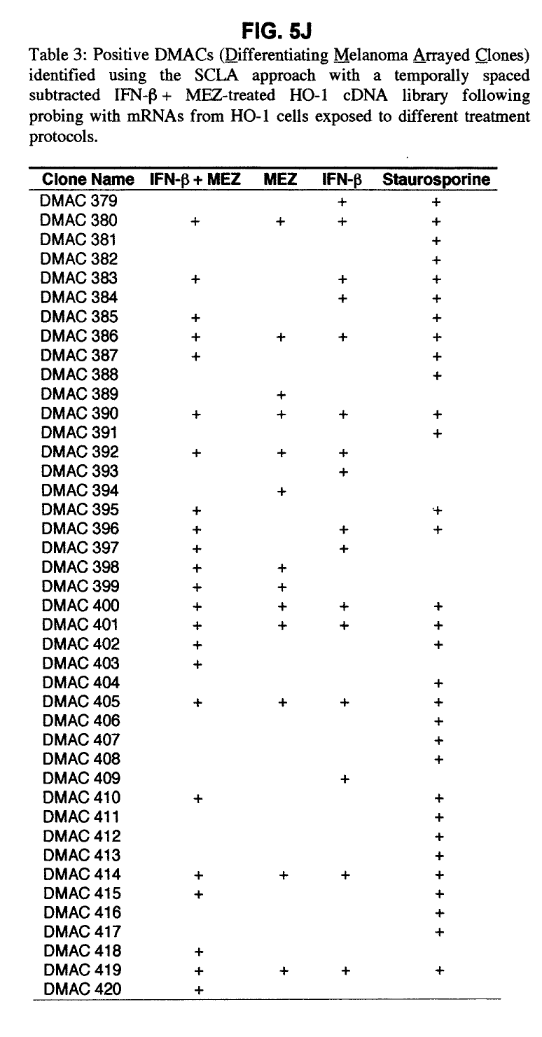

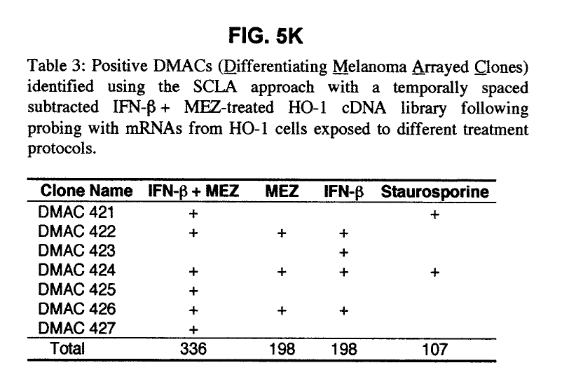

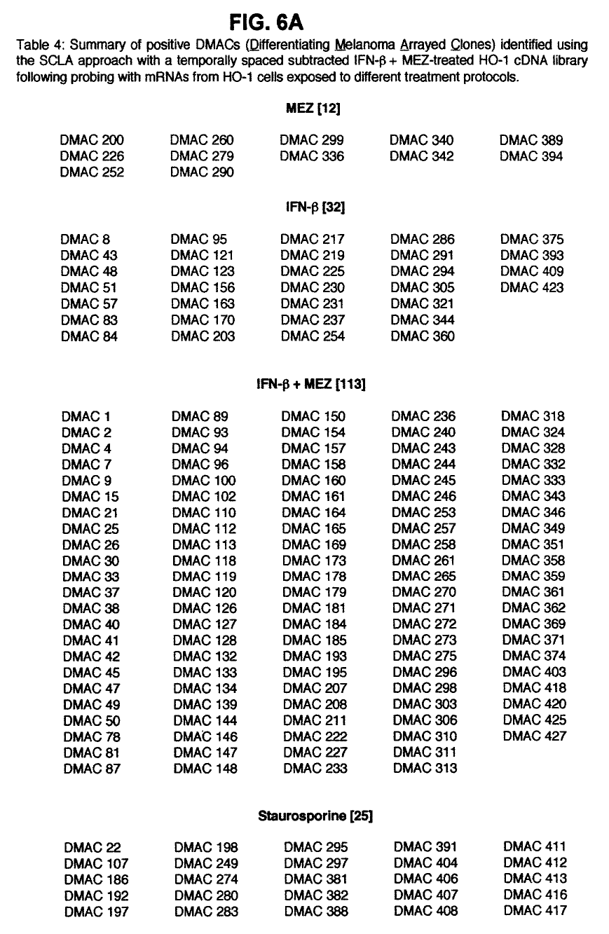

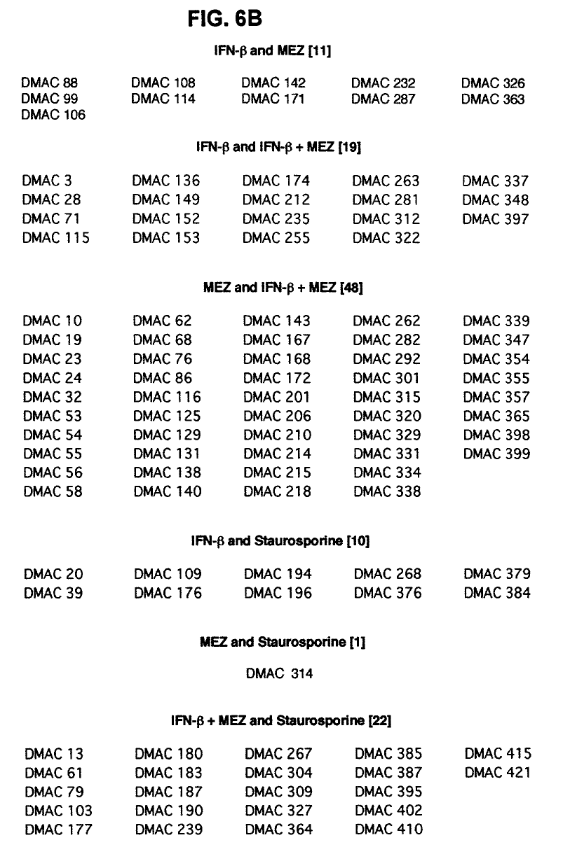

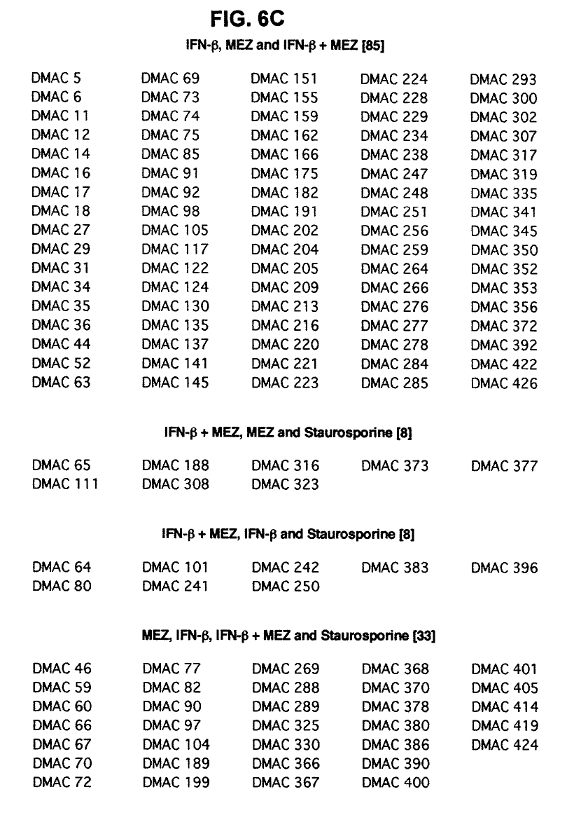

The SCLA approach represents a valuable strategy for systematically identifying and cloning cDNAs displaying differential expression as a function of treatment with specific agents modifying cellular physiology or in target cells displaying de novo differences in cellular phenotype, i.e., cancer versus normal, early versus late stages of development, chemotherapy sensitive versus chemotherapy resistant, etc. A schematic representation of the SCLA strategy, as used to identify genes displaying elevated expression as a function of treatment with specific modifiers of cellular physiology, is shown in FIG. 1. The SCLA approach has presently been used to test the hypothesis that induction of terminal cell differentiation in HO-1 cells results in the induced and/or elevated expression of unique and overlapping genes that may also be modulated as a consequence of reversible growth arrest, reversible differentiation and apoptosis. This scheme facilitates the identification of genes up-regulated by treatment with IFN-β+MEZ. [**To identify cDNAs down-regulated by IFN-β+MEZ the TESTER TS cDNA library would be prepared from untreated control HO-1 cells and the DRIVER TS cDNA library would be generated from IFN-β+MEZ treated HO-1 cells.] For the present application of SCLA, mRNAs were isolated after 2, 4, 8, 12, 24 and 48 hr from untreated control or IFN-β+MEZ (2000 units/ml+10 ng/ml) treated HO-1 cells. Subtraction hybridization was then performed as described in Jiang and Fisher (1993) in which the TESTER temporally spaced (TS) cDNA library was prepared from IFN-b+MEZ treated HO-1 cells and the DRIVER TS cDNA library was prepared from untreated HO-1 cells. Subtraction of DRIVER from TESTER at a 30 to 1 ratio resulted in ˜4,000 phage plaques. After subtraction hybridization, a temporally spaced amplified subtracted (TSS) cDNA library (consisting of 4,979 plaques) was picked and plated individually (arrayed) in thirteen 384-well microtiter plates. Bacteriophage were spotted on replicate Nylon filters and placed on bacterial lawns (allowing amplification of phage) thereby permitting subsequent probing by reverse Northern blotting using mRNAs isolated from HO-1 cells treated under various conditions, i.e., IFN-β+MEZ, IFN-β, MEZ or staurosporine. This approach permitted the identification of cDNAs displaying altered expression as a function of the specific treatment protocol. An example of the SCLA approach using replicate nylon membrane containing phage colonies sequentially probed with temporally pooled mRNAs from IFN-β, MEZ, IFN-β+MEZ and staurosporine treated HO-1 cells is shown in FIG. 2. Long exposure of autoradiograms indicate that ˜90% of plaques hybridize with an IFN-β+MEZ-treated cDNA probe, which demonstrates that the majority of arrayed phage contain DNA inserts. For this reason, only positive phage obtained after hybridization with IFN-β+MEZ mRNA (336 phage) and autoradiography for a short time period (2 hr) were considered positive for differential expression under this treatment protocol (Table 2).

Positive signals, theoretically representing differentially expressed cDNAs, can be isolated and analyzed, including sequence determination, temporal expression using Northern blotting, isolation of full-length cDNAs, etc. It has been documented that the majority of spotted phage that presumably contain cDNA inserts from the TSS IFN-β+MEZ HO-1 cDNA library will hybridize with IFN-β+MEZ treated HO-1 mRNAs (black circle indicating positive hybridization), specific independent and overlapping cDNAs will also hybridize with IFN-β, MEZ or staurosporine treated HO-1 mRNAs (black circle indicating positive hybridization) and very few cDNAs should hybridize with control HO-1 mRNAs (any positive hybridization, would suggest possible breakthrough in the subtraction procedure for specific cDNAs) (FIG. 1). The SCLA procedure directly identifies cDNAs potentially displaying an up-regulation of gene expression as a consequence of induction of growth suppression (IFN-β, MEZ, IFN-β+MEZ and staurosporine), reversible differentiation (IFN-β or MEZ), terminal differentiation (IFN-β+MEZ) or apoptosis (staurosporine and a small subset of cells treated with IFN-β+MEZ). Moreover, by reversing the direction of subtraction, i.e., TESTER cDNA is derived from the untreated HO-1 control cells and the DRIVER cDNA is obtained from the IFN-β+MEZ-treated HO-1 cells, SCLA can also be used to identify cDNAs down-regulated as a consequence of specific treatment protocols and the corresponding biochemical pathways that are modulated.

As summarized in Tables 2 to 4, a total of 427 positive clones, referred to as DMAC (Differentiating Melanoma Arrayed Clone) have been identified using the SCLA technology following probing with mRNAs isolated from IFN-β, MEZ, IFN-β+MEZ or staurosporine treated HO-1 cells. These include, pathway and agent specific (unique) clones, pathway and agent overlapping (shared) clones and clones potentially displaying antagonistic actions (competitive) (Tables 2 to 4). Of the 427 positive clones identified, 336 were identified after IFN-β+MEZ treatment, 198 were obtained following treatment with IFN-β, 198 were observed after MEZ treatment and 107 occurred after staurosporine treatment (Table 2). One hundred eighty two clones were uniquely positive after a single treatment protocol (IFN-β+MEZ, IFN-β, MEZ or staurosporine), whereas 245 clones displayed overlap with various treatment protocols (Tables 2 and 3). For example, specific clones were identified that were induced only by IFN-β (32), MEZ (12), IFN-β+MEZ (113) or staurosporine (25). Clones were recognized that overlapped following treatment with two agents, such as IFN-β and MEZ (11), MEZ and IFN-β+MEZ (48), IFN-βand IFN-β+MEZ (19), staurosporine and IFN-β+MEZ (22), staurosporine and MEZ (1) and staurosporine and IFN-β (10). A more complex overlapping induction pattern was also apparent with specific clones, including IFN-β, MEZ and IFN-β+MEZ (85), IFN-β+MEZ, MEZ and staurosporine (8), IFN-β+MEZ, IFN-β and staurosporine (8) and IFN-β+MEZ, IFN-β, MEZ and staurosporine (33). In contrast, no overlapping clones were apparent following treatment with IFN-β, MEZ and staurosporine.

Potential Functional Relevance of Agent and Pathway Specific Gene Expression Changes: Insights Based on the Proposed Mode of Action of Specific Inducing Agents

Although the identity of the 427 potentially differentially expressed DMACs remain to be determined, the pattern of their induction may provide valuable information relative to their connection with specific cellular changes and the pathways involved in induction. For example, IFN-β is a member of the interferon gene family that consists of a number of distinct and related secreted proteins that elicit multiple effects in specific target cells, including growth suppression, changes in differentiation and immunologic alterations (including antigenic modulation) (Fisher and Grant, 1985; Pestka et al., 1987; Haque and Williams, 1998; Pfeffer et al., 1998; Stark et al., 1998; Roberts et al., 1999a). Interferon alpha (IFN-α) and IFN-β are classified as type I interferons that interact with the type I interferon receptor. After binding to its cell surface receptor IFN-a and IFN-β activate the transcription of a diverse set of genes, many of which contain specific enhancer sequences in their promoter region called the interferon stimulated response element (ISRE). The activation of changes in gene expression by type I interferons involves the Janus family of kinases (Jaks) and the signal transduction activators of transcription (Stats) (Haque and Williams, 1998; Pfeffer et al., 1998; Stark et al., 1998; Roberts et al., 1999a). By means of the Jak/Stat proteins, signals are transmitted from the interferon receptors on the cell surface to the nucleus thereby mediating the function of various cytokines, hormones and growth factors. Activation of specific Jak/Stat pathways can also inhibit growth and modify programs of differentiation in specific target cell types. In this context, DMACs induced solely by IFN-β (32 DMACs) and IFN-β and IFN-β+MEZ (19 DMACs), are most likely activated as a consequence of this well-defined pathway. By sub-arraying the 32 DMACs uniquely induced by IFN-β and 19 DMACs uniquely induced by IFN-β and IFN-β +MEZ and probing with mRNAs from different cell types or treatment conditions, it is possible to obtain important insights into factors contributing to the regulation of expression of these cDNAs. For example, it is possible to determine if any of these DMACs are also modulated in additional cell types treated with IFN-β or IFN-β+MEZ, i.e., are they HO-1 or melanoma specific or are they type I interferon specific. One could test if treatment of HO-1 or other cell types with IFN-γ or additional cytokines induce any of these DMACs. A reverse Northern hybridization approach could also determine if chemotherapeutic agents can induce any of these DMACs. In these contexts, significant information can be obtained relative to a specific DMAC without having prior sequence information, an intrinsic property of the SCLA approach. In addition, since it is assumed, that the Jak/Stat pathway is directly associated with activation of IFN-β (unique) and IFN-β and IFN-β+MEZ (unique)-inducible DMACs, up-regulation of any of these DMACs during cellular processes such as senescence, DNA damage, development, chemotherapy, etc., would indirectly implicate activation of the Jak/Stat pathway in these phenomena. This hypothesis is readily testable.

Mezerein (MEZ) is a daphnane diterpene with unsaturated side chains that belongs to a group of second-stage tumor promoters. MEZ preferentially activates the alpha subtype of serine/threonine-specific protein kinase C (PKC), PKC-α (Geiges et al., 1997). PKC is the major receptor for a number of tumor-promoting agents that utilizes 1,2 -diacylglycerol (DAG) and/or additional lipids to modulate various cellular functions, including exocytosis, gene expression, proliferation, differentiation and tumor promotion (Nishizuka, 1992; Borner and Fabbro, 1992; Hug and Sarre, 1994). Staurosporine is a potent PKC inhibitor effecting the activity of numerous PKC subtypes (Tamaoki et al., 1986; Geiges et al., 1997). In addition, staurosporine is also a potent inducer of apoptosis in HO-1 and other cell types. In these contexts, the 12 DMACs induced uniquely by MEZ and 48 DMACs induced uniquely by MEZ or the combination of IFN-β+MEZ in HO-1 cells implicate PKC activation and associated biochemical changes as potential contributors to altered gene expression. Since staurosporine inhibits PKC-A activation, a link between activation of PKC-A and up-regulation of these 60 DMACs can easily be investigated by treating HO-1 cells with a combination of these agents and evaluating the effect on gene up-regulation. Moreover, since PKC is inhibited by staurosporine and treated cells undergo programmed cell death (apoptosis), the 25 DMACs specifically up-regulated by staurosporine may represent important components of this process in mammalian cells. Sequence information for the staurosporine specific DMACs will be valuable in determining if these genes are novel or if they represent previously recognized contributors to apoptosis. The staurosporine specific DMACs can be sub-arrayed and directly evaluated for their relationship to apoptosis. This could include, an evaluation of expression as a function of treatment with additional agents inducing apoptosis in HO-1 cells, after staurosporine exposure of diverse cell types and after treatment with agents eliciting necrotic cell death or toxicity.

A major degree of complexity arises when a specific DMAC is modulated by multiple agents or in situations where one treatment protocol may inhibit up-regulation of a DMAC, such as IFN-β or MEZ only inducible genes that are not actively up-regulated in the IFN-β+MEZ subtracted arrayed cDNA library. In a situation where the mode of action of the inducers is different, such as IFN-β and MEZ, a DMAC that is induced by both of these treatments suggests that multiple biochemical pathways can activate the same gene. In cases where the combination of IFN-β+MEZ and either IFN-β (19 DMACs) or MEZ (48 DMACs) alone specifically induce the same DMAC, this would suggest that a specific pathway, i.e., Jak/Stat for IFN-β or PKC for MEZ, may predominate when IFN-β and MEZ are used in combination. For DMACs activated by both IFN-β+MEZ and staurosporine, similar biochemical changes and pathways may be elicited during the processes of terminal differentiation and apoptosis. Eighty five discrete DMACs are activated when HO-1 cells are treated with IFN-β, MEZ or IFN-β+MEZ. In this context, these DMACs may represent gene changes that are associated with the different components of the growth arrest and differentiation process induced by the different treatment protocols. The highest level of complexity of regulation may involve the 33 DMACs that display elevated expression as a function of IFN-β, MEZ, IFN-β+MEZ and staurosporine treatment. These changes may reflect gene pathways that overlap with either the specific chemical employed or reflect commonality in changes induced by these agents. A clearer perspective on the potential role and association between the various DMACs and the treatment protocols and pathways induced will be obtained after sequencing and further testing of the 427 DMACs.

Applications of the SCLA Approach for Defining Complex Programs of Gene Expression and Cloning Differentially Expressed Genes

The SCLA strategy will prove of immense value for multiple applications in identifying and cloning genes involved in specific physiological changes in cells. This scheme will also provide utility for defining the molecular determinants associated with and potentially controlling diverse biochemical alterations in cells, tissues and organisms. Some potential applications of SCLA include:

(a) Defining Gene Expression Changes and Identifying and Cloning Genes Unique to Cancer or Normal Cells.

To identify gene specific changes and identify and clone genes associated with cancer, cDNA libraries can be constructed from pooled multiple primary (microdissected) tumor samples, multiple metastatic lesions or multiple human tumor cell lines. Corresponding cDNA libraries can be prepared from pooled normal counterparts, i.e., primary normal tissue or normal cell lines. Subtraction hybridization can then be performed, TESTER (tumor)—DRIVER (normal) producing a subtracted tumor-enriched (STE) cDNA library or TESTER (normal)—DRIVER (tumor) generating a subtracted normal-enriched (SNE) cDNA library. The STE and SNE cDNA libraries can then be arrayed, as described for the currently described SCLA approach or using a robotic setup to facilitate isolation of potential cDNA containing phage, for SCLA analysis. The STE transferred arrayed clones can be tested using mRNAs isolated from similar or different tumor subtypes to identify cDNAs that represent cancer subtype and more ubiquitous cancer specific genes. The SNE transferred arrayed clones can be tested using mRNAs isolated from similar or different normal subtypes to identify cDNAs that represent genes displaying elevated expression in normal versus cancer cells. The genes that are identified can be used for diagnostic and potentially prognostic applications. If functionally involved in the cancer phenotype, specific genes may also provide therapeutic applications.

(b) Defining Gene Expression Chances and Identifying and Cloning Genes Associated with Embryonic Development.

To identify gene changes and to identify and clone genes that specifically associate with embryonic and fetal development and that are not apparent, or display reduced activity, in the adult tissue or organism, cDNA libraries can be constructed from different tissue of an adult and different temporal points in the developing embryo and fetus. Subtraction hybridization can then be performed, TESTER (embryonic or fetal organ)—DRIVER (adult organ) resulting in a subtracted embryo/fetus organ-enriched (E/FOE) cDNA library. The E/FOE cDNA library can then be arrayed, as described for the currently described SCLA approach or using a robotic setup to facilitate isolation of potential cDNA containing phage, for SCLA analysis. The E/FOE arrayed clones can be tested using specific temporal mRNAs isolated from different staged embryo and fetal organs to define genes directly regulated during development. These genes can be used to monitor for normal development and may have therapeutic intervention applications for correcting genetically or environmentally induced fetal abnormalities.

(c) Defining Gene Expression Chances and Identifying and Cloning Genes Associated with Chemotherapy Resistance.

To identify gene changes and to identify and clone genes specifically associated with chemotherapy resistance, cDNA libraries can be constructed from cells refractive and sensitive to toxicity or apoptosis induced by specific therapeutic agents, including chemical (dauxorubicin, cisplatin, taxol, taxotere, etc.) and physical (such as irradiation) treatment protocols. Subtraction hybridization can then be performed, TESTER (resistant)—DRIVER (sensitive) producing a subtracted chemotherapy resistance enriched (CRE) cDNA library or TESTER (sensitive)—DRIVER (resistant) generating a subtracted chemotherapy sensitive enriched (CSE) cDNA library. The CRE and CSE cDNA libraries can then be arrayed, as described for the currently described SCLA approach or using a robotic setup to facilitate isolation of potential cDNA containing phage, for SCLA analysis. The arrayed cDNA libraries can be probed with mRNAs from a spectrum of chemotherapy resistant and sensitive tumor cell types to identify genes that specifically associate with resistance or sensitivity to specific therapeutic protocols. The genes that are identified can be used for diagnostic and potentially prognostic applications. If functionally involved in the resistance or sensitive phenotypes, specific genes may also provide therapeutic applications.

(d) Defining Gene Expression Changes and Identifying and Cloning Genes Modified during Chemotherapy.

To identify gene changes and to identify and clone genes specifically modulated by treatment with chemotherapeutic agents, temporal cDNA libraries can be constructed from cells un-treated or treated with specific therapeutic agents, including chemical (dauxorubicin, cisplatin, taxol, taxotere, etc.) and physical (such as irradiation) treatment protocols. Subtraction hybridization can then be performed, TESTER (therapy treated)—DRIVER (therapy un-treated) producing a temporal subtracted therapy treated enriched (TTE) cDNA library or TESTER (therapy un-treated)—DRIVER (therapy treated) generating a temporal subtracted cherapy un-treated enriched (TU-TE) cDNA library. The TTE and TU-TE cDNA libraries can then be arrayed, as described for the currently described SCLA approach or using a robotic setup to facilitate isolation of potential cDNA containing phage, for SCLA analysis. The arrayed cDNA libraries can be probed with mRNAs from a spectrum of tumor cell types either treated or un-treated with specific therapeutic reagents or protocols to identify genes that specifically associate with response or lack of response to the therapeutic protocols. The genes that are identified can be used for diagnostic and potentially prognostic applications. If functionally involved in the therapeutic or lack of therapeutic response, specific genes may also provide therapeutic applications.

(e) Defining Gene Expression Changes and Identifying and Cloning Genes Involved in Cellular Senescence.

To identify gene changes and to identify and clone genes specifically associated with cellular senescence, cDNA libraries can be constructed from senescent and non-senescent cell cultures. Subtraction hybridization can then be performed, TESTER (senescent)—DRIVER (non-senescent) resulting in a subtracted senescence enriched (SE) cDNA library or TESTER (non-senescent)—DRIVER (senescent) producing a subtracted non-senescent enriched (N-SE) cDNA library. The SE and N-SE cDNA libraries can then be arrayed, as described for the currently described SCLA approach or using a robotic setup to facilitate isolation of potential cDNA containing phage, for SCLA analysis. The arrayed cDNA libraries can be probed with mRNAs from a spectrum of senescent and non-senescent cultures to identify genes that specifically associate with senescence and non-senescence. The genes that are identified can be used for diagnostic and potentially prognostic applications. If functionally involved in senescence, specific genes may also have therapeutic applications.

(f) Defining Gene Expression Changes and Identifying and Cloning Genes Involved in Apoptosis.

To identify gene expression changes and to identify and clone genes specifically associated with apoptosis, temporally spaced cDNA libraries can be constructed from apoptotic and non-apoptotic cell cultures. Subtraction hybridization can then be performed, TESTER (apoptotic)—DRIVER (non-apoptotic) producing a subtracted apoptosis enriched (AE) cDNA library or TESTER (non-apoptotic)—DRIVER (apoptotic) generating a subtracted non-apoptosis enriched (N-AE) cDNA library. To define the complete repertoire of genes potentially involved in apoptosis, temporally spaced subtracted cDNA libraries are preferable to a single time point subtracted library. The AE and N-AE cDNA libraries can then be arrayed, as described for the currently described SCLA approach or using a robotic setup to facilitate isolation of potential cDNA containing phage, for SCLA analysis. The arrayed cDNA libraries can be probed with temporally spaced mRNAs from apoptotic and non-apoptotic cultures to identify genes that may induce apoptosis (pro-apoptotic) and inhibit apoptosis (anti-apoptosis). The genes that are identified can be used for diagnostic and potentially prognostic applications. If functionally involved in apoptosis, specific genes may also provide therapeutic applications.

(g) Defining Gene Expression Chances and Identifying and Cloning Genes Involved in Terminal Cell Differentiation.

To identify gene expression changes and to identify and clone genes specifically associated with terminal differentiation in different model systems, temporally spaced cDNA libraries can be constructed from terminally differentiated and non-differentiating cell cultures. Subtraction hybridization can then be performed, TESTER (terminally differentiated)—DRIVER (non-differentiating) resulting in a subtracted terminal differentiation enriched (TDE) cDNA library or TESTER (non-differentiating)—DRIVER (terminally differentiated) generating a subtracted non-differentiating enriched (N-DE) cDNA library. The TDE and N-DE cDNA libraries can then be arrayed, as described for the currently described SCLA approach or using a robotic setup to facilitate isolation of potential cDNA containing phage, for SCLA analysis. The arrayed cDNA libraries can be probed with mRNAs from temporally spaced differentiating and non-differentiating cultures to identify genes that may are up-regulated (subtracted TDE cDNA library) or down-regulated (subtracted N-DE cDNA library). The genes that are identified can be used for diagnostic and potentially prognostic applications. If functionally involved in growth control and terminal differentiation, specific genes may also result in therapeutic applications.

(h) Defining Gene Expression Changes and Identifying and Cloning Genes Involved in Neurodegeneration.

To identify gene expression changes and to identify and clone genes specifically associated with neurodegeneration, temporally or non-temporally spaced, depending on the treatment protocol, cDNA libraries can be constructed from normal (non-neurodegenerative) control and cultures displaying or induced to undergo neurodegeneration by chemical treatment, by virus, as a consequence of undefined genetic abnormalities, during aging, etc. Subtraction hybridization can then be performed, TESTER (neurodegeneration positive)—DRIVER (neurodegeneration negative) producing a subtracted neurodegeneration positive enriched (NPE) cDNA library or TESTER (neurodegeneration negative)—DRIVER (neurodegeneration positive) forming a subtracted neurodegeneration negative enriched (NNE) cDNA library. The NPE and NNE cDNA libraries can then be arrayed, as described for the currently described SCLA approach or using a robotic setup to facilitate isolation of potential cDNA containing phage, for SCLA analysis. The arrayed cDNA libraries can be probed with mRNAs from neurodegenerative conditions (various disease states (Alzheimer's, Huntington's chorea, Parkinson's, amyotrophic lateral sclerosis, etc.), HIV-1 infected, chemical treated, aged, etc.) and their normal counterparts to identify genes that are associated with neruodegenerative changes. The genes that are identified can be used for diagnostic and potentially prognostic applications. If functionally involved in neurodegeneration, specific genes may also have therapeutic applications.

(i) Defining Gene Expression Changes and Identifying and Cloning Genes Involved in Cardiac Dysfunction.

To identify gene expression changes and to identify and clone genes specifically associated with cardiac dysfunction, cDNA libraries can be constructed from cardiac tissue displaying dysfunction and normal cardiac tissue. Subtraction hybridization can then be performed, TESTER (cardiac dysfunction tissue)—DRIVER (normal cardiac tissue) resulting in a subtracted cardiac dysfunction tissue enriched (CDTE) cDNA library or TESTER (normal cardiac tissue)—DRIVER (cardiac dysfunction tissue) producing a subtracted normal cardiac tissue enriched (NCTE) cDNA library. The CDTE and NCTE cDNA libraries can then be arrayed, as described for the currently described SCLA approach or using a robotic setup to facilitate isolation of potential cDNA containing phage, for SCLA analysis. The arrayed cDNA libraries can be probed with mRNAs from cardiac dysfunction and normal cardiac tissue to identify genes that may be up-regulated during cardiac dysfunction CDTE cDNA library) or down-regulated during cardiac dysfunction (NCTE cDNA library). The genes that are identified can be used for diagnostic and potentially prognostic applications. If functionally involved in cardiac dysfunction, specific genes may also have therapeutic applications.

(j) Defining Gene Expression Chances and Identifying and Cloning Genes Involved in angiogenesis.

To identify gene expression changes and to identify and clone genes specifically associated with angiogenesis, cDNA libraries can be constructed from angiogenic tumors, poorly angiogenic tumors and angiogenesis induced and un-induced cultures. Subtraction hybridization can then be performed, TESTER (angiogenic state)—DRIVER (poor/non-angiogenic state) resulting in a subtracted angiogenesis enriched (AgE) cDNA library or TESTER (poor/non-angiogenic state)—DRIVER (angiogenic state) generating a subtracted poor/non-angiogenic enriched (P/N-AgE) cDNA library. The AgE and P/N-AgE cDNA libraries can then be arrayed, as described for the currently described SCLA approach or using a robotic setup to facilitate isolation of potential cDNA containing phage, for SCLA analysis. The arrayed cDNA libraries can be probed with mRNAs from various samples displaying high or low angiogenesis properties to identify genes that may be up-regulated (subtracted AE cDNA library) or down-regulated (subtracted P/N-AE cDNA library). The genes that are identified can be used for diagnostic and potentially prognostic applications. If functionally involved in angiogenesis, specific genes may also prove useful for therapeutic applications.

(k) Defining Gene Expression Chances and Identifying and Cloning Genes Involved in Muscular Degeneration.

To identify gene expression changes and to identify and clone genes specifically associated with muscular degeneration, cDNA libraries can be constructed from muscle tissue displaying degeneration and normal muscle tissue. Subtraction hybridization can then be performed, TESTER (muscle degeneration tissue)—DRIVER (normal muscle tissue) generating a subtracted muscle degeneration tissue enriched (MDTE) cDNA library or TESTER (normal muscle tissue)—DRIVER (muscle degeneration tissue) producing a subtracted normal muscle tissue enriched (NMTE) cDNA library. The CDTE and NCTE cDNA libraries can then be arrayed, as described for the currently described SCLA approach or using a robotic setup to facilitate isolation of potential cDNA containing phage, for SCLA analysis. The arrayed cDNA libraries can be probed with mRNAs from muscle tissue undergoing degeneration and normal muscle tissue to identify genes that may be up-regulated during muscle degeneration (MDTE cDNA library) or down-regulated during cardiac dysfunction (NMTE cDNA library). The genes that are identified can be used for diagnostic and potentially prognostic applications. If functionally involved in muscle degeneration, specific genes may also provide therapeutic applications.

(1) Defining Gene Expression Changes and Identifying and Cloning Genes Involved in Cellular and Tissue Response to Infectious Agents.

To identify gene expression changes and to identify and clone genes specifically associated with infection (virus, bacteria, fungus or parasite), cDNA libraries can be constructed from infected cells and tissues displaying dysfunction and normal non-infected cells and tissues. Subtraction hybridization can then be performed, TESTER (infected tissue)—DRIVEP (normal non-infected tissue) producing a subtracted infected tissue enriched (ITE) cDNA library or TESTER (normal non-infected tissue)—DRIVER (infected tissue) resulting in a subtracted normal non-infected tissue enriched (NN-ITE) cDNA library. The ITE and NN-ITE cDNA libraries can then be arrayed, as described for the currently described SCLA approach or using a robotic setup to facilitate isolation of potential cDNA containing phage, for SCLA analysis. The arrayed cDNA libraries can be probed with mRNAs from infected (virus, bacteria, fungus or parasite) and normal non-infected cells and tissues to identify genes that are up-regulated during infection with the specific agent (ITE cDNA library) or down-regulated during infection with a specific agent (NN-ITE cDNA library). The genes that are identified can be used for diagnostic and potentially prognostic applications. If functionally involved in the infection process, specific genes may also provide therapeutic applications.

A methodology, Sequential cDNA Library Array (SCLA), is described that permits the identification and cloning of genes displaying differential expression as a consequence of induction of growth suppression, reversible differentiation, terminal differentiation and apoptosis. This scheme has wide applicability for systematically evaluating and identifying cDNAs in a subtracted library displaying differential expression as a function of complex changes in cell physiology. The SCLA approach will be beneficial for studying a large number of intricate biological processes and it offers potential for identifying and cloning genes providing diagnostic, prognostic and therapeutic utility.

What is claimed is:

1. A method of producing a sequential cDNA library array comprising:
a) obtaining a subtracted cDNA library having clones containing cDNA inserts and said library also allows propagation of the cDNA inserts;
b) arraying the cDNA library into individual clone such that each clone can propagate on its own;
c) transferring the arrayed clones onto a solid matrix thereby generating a cDNA library replica;
d) extracting the nucleic acid from the clone; and
e) generating a subarray of phage clones or DNA inserts.

2. A method of claim 1, wherein more than one array is produced.

3. The method of claim 1, wherein the solid matrix in step 1(c) is a membrane.

4. The method of claim 1, wherein the solid matrix is nylon, nitrocellulose, glass, plastic or DEAE-cellulose.

5. The method of claim 1, wherein the cDNA library is a λZAP subtracted cDNA library.

6. The method of claim 1, wherein the cDNA library is any subtracted cDNA library.

7. A method of identifying genes comprising hybridizing the cDNA library array resulting from claim 1 with specific probes.

8. The method of claim 7, wherein the probes are RNA or DNA.

9. The method of claim 7, wherein the specific probes are derived from target cells, tissues, organs, or an organism.

10. The method of claim 9, wherein the target cells are cancerous.

11. The method of claim 9, wherein the target cells are normal.

12. The method of claim 9 wherein the target cells are embryonic cells, adult cells, infected cells or drug treated cells.

13. The method of claim 7, wherein the specific probes are derived from a treatment inducing a specific cellular change or pathway.

14. The method of claim 13, wherein the cellular change or pathway is growth regulation, terminal differentiation, apoptosis, senescence, neurodegeneration, cardiac dysfunction, angiogenesis or muscular degeneration.

15. The method of claim 13, wherein the pathway or cellular change is treatment induced.

16. The method of claim 13, wherein treatment is with a infectious agent or a chemotherapeutic agent.

17. The method of claim 16, wherein the infectious agent is a virus, bacteria, fungus or parasite.

18. The method of claim 13, wherein the pathway or cellular change is a biochemical pathway.

19. The method of claim 13, wherein the pathway or cellular change is induced by environmental changes or therapeutic changes.

20. The method of claim 1, wherein the subtracted cDNA library is derived from the HO-1 melanoma cells.

21. The method of claim 20, wherein the melanoma cells are HO-1 melanoma cells treated with IFN-β plus MEZ.

22. The method of claim 20, wherein the cells are untreated HO-1 melanoma cells.

23. A gene identified by the method of claim 7.

24. An isolated nucleic acid molecule designated Differentiating Melanoma Arrayed Clone (DMAC)-1 thru DMAC-427.

25. A protein encoded by the gene of claim 7.

26. An antibody capable of specifically recognizing the protein of claim 25.

27. The method of claim 1, wherein the sequential cDNA library array is used for diagnosis, genetic screening or prognosis.

28. The method of claim 27, wherein the diagnosis, genetic screening or prognosis is between cell or tissue types.

29. The method of claim 27, wherein the diagnosis, genetic screening or prognosis is between species.
